(19) United States
(12) Patent Application Publication
(10) Pub. No.: US 2016/0084718 A1
(43) Pub. Date: Mar. 24, 2016

(54) DEVICE AND METHOD FOR TEMPERATURE DETECTION AND MEASUREMENT USING INTEGRATED COMPUTATIONAL ELEMENTS

(71) Applicant: Halliburton Energy Services Inc., Houston, TX (US)

(72) Inventor: David Warren TEALE, Spring, TX (US)

(21) Appl. No.: 14/787,069

(22) PCT Filed: Jun. 20, 2013
(86) PCT No.: PCT/US2013/046883

§ 371 Date: Oct. 26, 2015

(51) Int. Cl. G01K 11/00; G01N 33/24

(52) U.S. Cl. CPC G01K 11/00; G01N 33/241

(57) ABSTRACT

An optical computing device and method for determining and/or monitoring temperature and temperature variation data in real-time by deriving the data from the output of an optical element.

### FIELD OF THE INVENTION

The present invention relates generally to optical sensors and, more specifically, to an Integrated Computational Element (“ICE”) based optical device for real-time temperature detection and measurement in a variety of environments.

### BACKGROUND

Temperature is measured in many industries for a variety of reasons. One such industry is hydrocarbon exploration and recovery. Conventionally, downhole temperature measurement falls into one of three distinct categories: electronic pressure/temperature gauge-based sensing, fiber-based distributed temperature sensing, and thermal couple-based sensing.

However, these conventional temperature measurement techniques are disadvantageous due to their power, communication and space requirements. For example, each requires deployment of support and auxiliary hardware and support systems including down-hole mandrel assemblies for protection and mounting of sensing hardware, dedicated power sources or architecture to supply power and communications, and surface equipment to support data management. Moreover, traditional devices are typically stand-alone systems and are not readily incorporated into other downhole tools.

Accordingly, there is a need in the art for a cost-effective, compact and power efficient system in which to detect and monitor real-time temperature data in a desired environment.

### BRIEF DESCRIPTION OF THE DRAWINGS

FIG. 1 illustrates a well system having an optical computing device deployed therein for temperature detection according to certain exemplary embodiments of the present invention;

FIG. 2 is a block diagram of an optical computing device employing a transmission mode design for temperature detection, according to certain exemplary embodiments of the present invention;

FIG. 3 is a block diagram of another optical computing device employing a time domain mode design for temperature detection, according to certain exemplary embodiments of the present invention; and

FIG. 4 is a flow chart of a temperature detection methodology performed by an optical computing device in accordance to certain exemplary methods of the present invention.

### DESCRIPTION OF ILLUSTRATIVE EMBODIMENTS

Illustrative embodiments and related methodologies of the present invention are described below as they might be employed in an optical computing device and method to determine the temperature of a sample in a variety of environments. In the interest of clarity, not all features of an actual implementation or methodology are described in this specification. Also, the “exemplary” embodiments described herein refer to examples of the present invention. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. Further aspects and advantages of the various embodiments and related methodologies of the invention will become apparent from consideration of the following description and drawings.

Exemplary embodiments of the present invention are directed to an optical computing device that determines and monitors temperature and temperature variation data in real-time by deriving the data directly from the output of an optical element (ICE, for example). In certain embodiments, the optical computing device is a dedicated, single-purpose device that obtains temperature data, while in other embodiments the optical computing device acts as a dual-purpose device that obtains temperature data and various other characteristic data of the measured sample. In either embodiment, the present invention determines the temperature of a sample/environment based on the physical and/or optical responses of the sample to temperature changes. In the dual purpose embodiment, the temperature data is derived as a secondary function of the optical computing device and does not interfere with its primary mode of operation (i.e., detecting characteristic data). Accordingly, the present invention provides real-time temperature monitoring in a variety of space-limited or power constrained environments.

In the most preferred embodiment, the optical computing devices described herein utilize one or more ICEs (also known as a Multivariate Optical Element (“MOE”)) as the optical elements. Alternatively, however, narrow band filters may also be utilized as the optical elements. Nevertheless, as will be understood by those ordinarily skilled in the art having the benefit of this disclosure, an ICE is an optical element configured to receive an input of electromagnetic radiation from a substance or sample of the substance and produce an output of electromagnetic radiation that corresponds to a characteristic of the sample. Fundamentally, optical computing devices utilize the ICE to perform calculations, as opposed to the hardwired circuits of conventional electronic processors. When electromagnetic radiation interacts with a substance, unique physical and chemical information about the substance is encoded in the electromagnetic radiation that is reflected from, transmitted through, or radiated from the sample. Thus, the optical computing device, through use of the ICE, is capable of extracting the information of one or multiple characteristics/properties or analytes within a sample, and converting that information into a detectable output regarding the overall properties of a sample.

Further discussion of the design and operation of ICEs and optical computing devices can be found in, for example, U.S. Pat. No. 6,198,531, entitled “OPTICAL COMPUTATIONAL SYSTEM,” issued to Myrick et al. on Mar. 6, 2001; U.S. Pat. No. 7,697,141, entitled “IN SITU OPTICAL COMPUTATION FLUID ANALYSIS SYSTEM AND METHOD,” issued to Jones et al. on Apr. 13, 2010; and U.S. Pat. No. 8,049,881, entitled “OPTICAL ANALYSIS SYSTEM AND METHODS FOR OPERATING MULTIVARIATE OPTICAL ELEMENTS IN A NORMAL INCIDENCE ORIENTATION,” issued to Myrick et al. on Nov. 1, 2011, each being owned by the Assignee of the present invention, Halliburton Energy Services, Inc., of Houston, Tex., the disclosure of each being hereby incorporated by reference in its entirety.

As further described herein, the exemplary optical computing devices determine temperature through utilization of the unique physical and chemical information encoded in the radiation emanating from the sample. As will be understood by those ordinarily skilled in the art having the benefit of this disclosure, the sample and optical computing device behaves according to laws of physics and are typically calibrated to remove unwanted noise and environmental effects, such as, for example, temperature, pressure, stress, or electrical phenomena. It is this principle—that all materials undergo change due to external effects (temperature, for example)—which allows the present inventive optical computing device to measure the corresponding effect (temperature). In other words, the present invention collects spectral information as a function of physical variables of the sample or optical element. Thus, as the physical variables are altered due to temperature changes, there is a corresponding shift in the spectral information relative to the baseline data. Therefore, the present invention analyzes the baseline shift in the spectral information to determine the corresponding temperature.

The optical computing devices described herein may be utilized in a variety of environments. Such environments may include, for example, downhole well or completion applications. Other environments may include those as diverse as those associated with surface and undersea monitoring, satellite or drone surveillance, pipeline monitoring, or even sensors transiting a body cavity such as a digestive tract. Within those environments, the optical computing devices are utilized to detect and monitor temperature, in addition to detecting various compounds or characteristics in order to monitor, in real time, various phenomena occurring within the environment.

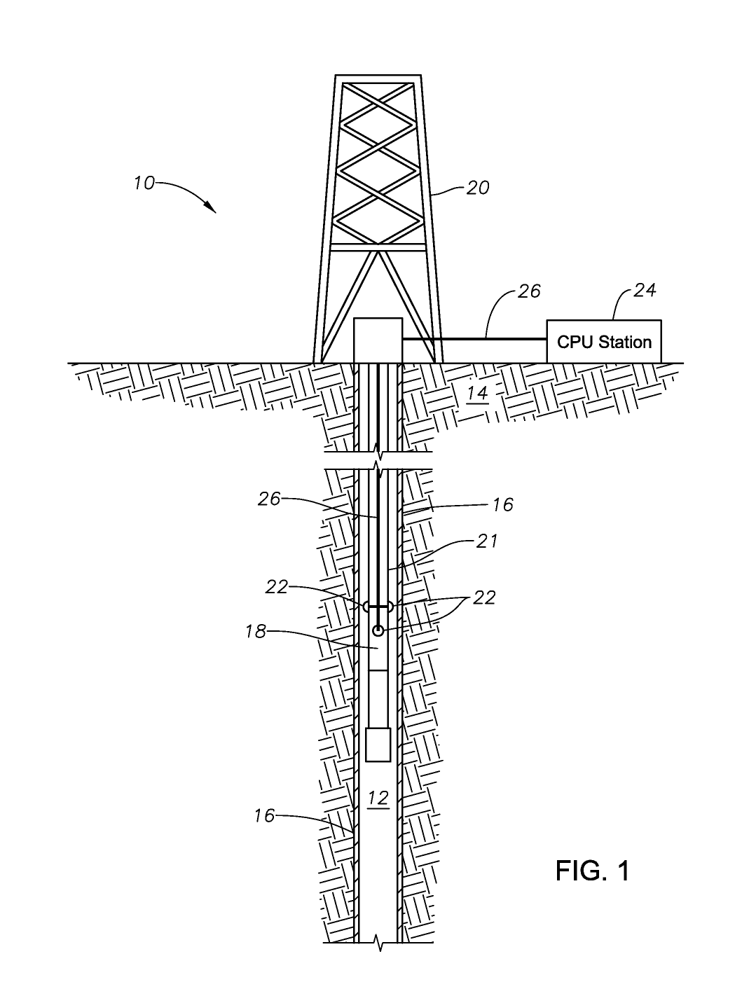

Although the optical computing devices described herein may be utilized in a variety of environments, the following description will focus on downhole well applications. FIG. 1 illustrates a plurality of optical computing devices 22 positioned along a workstring 21 extending along a downhole well system 10 according to certain exemplary embodiments of the present invention. Workstring 21 may be, for example, a logging assembly, production string or drilling assembly. Well system 10 comprises a vertical wellbore 12 extending down into a hydrocarbon reservoir 14 (although not illustrated, wellbore 12 may also comprise one or more lateral sections). Wellbore equipment 20 is positioned atop vertical wellbore 12, as understood in the art. Wellbore equipment may be, for example, a blow out preventer, derrick, floating platform, etc. As understood in the art, after vertical wellbore 12 is formed, tubulars 16 (casing, for example) are extended therein to complete wellbore 12.

One or more optical computing devices 22 may be positioned along wellbore 12 at any desired location. In certain embodiments, optical computing devices 22 are positioned along the internal or external surfaces of downhole tool 18 (as shown in FIG. 1) which may be, for example, intervention equipment, surveying equipment, or completion equipment including valves, packers, screens, mandrels, gauge mandrels, in addition to casing or tubing tubulars/joints as referenced below. Alternatively, however, optical computing devices 22 may be permanently or removably attached to tubulars 16 and distributed throughout wellbore 12 in any area in which temperature detection/monitoring is desired. Optical computing devices 22 may be coupled to a remote power supply (located on the surface or a power generator positioned downhole along the wellbore, for example), while in other embodiments each optical computing device 22 comprises an on-board battery. Moreover, optical computing devices 22 are communicably coupled to a CPU station 24 via a communications link 26, such as, for example, a wireline or other suitable communications link. Those ordinarily skilled in the art having the benefit of this disclosure will readily appreciate that the number and location of optical computing devices 22 may be manipulated as desired.

Each optical computing device 22 comprises an ICE that optically interacts with a sample of interest (wellbore fluid, downhole tool component, tubular, for example) to determine the temperature of the sample, and thus the temperature of the surrounding environment. In certain exemplary embodiments, optical computing devices 22 may be dedicated to temperature detection or, alternatively, they may serve the dual-purpose of sample temperature and characteristic detection. In the latter embodiments, exemplary characteristics determined by optical computing devices 22 include the presence and quantity of specific inorganic gases such as, for example, CO₂ and H₂S, organic gases such as methane (C1), ethane (C2) and propane (C3) and saline water, in addition to dissolved ions (Ba, Cl, Na, Fe, or Sr, for example) or various other characteristics (p.H., density and specific gravity, viscosity, total dissolved solids, sand content, etc.). In certain embodiments, a single optical computing device 22 may detect a single characteristic, while in others a single optical computing device 22 may determine multiple characteristics, as will be understood by those ordinarily skilled in the art having the benefit of this disclosure.

CPU station 24 comprises a signal processor (not shown), communications module (not shown) and other circuitry necessary to achieve the objectives of the present invention, as will be understood by those ordinarily skilled in the art having the benefit of this disclosure. In addition, it will also be recognized that the software instructions necessary to carry out the objectives of the present invention may be stored within storage located in CPU station 24 or loaded into that storage from a CD-ROM or other appropriate storage media via wired or wireless methods. Communications link 26 provides a medium of communication between CPU station 24 and optical computing devices 22. Communications link 26 may be a wired link, such as, for example, a wireline or fiber optic cable extending down into vertical wellbore 12. Alternatively, however, communications link 26 may be a wireless link, such as, for example, an electromagnetic device of suitable frequency, or other methods including acoustic communication and like devices.

In certain exemplary embodiments, CPU station 24, via its signal processor, controls operation of each optical computing device 22. In addition to sensing operations, CPU station 24 may also control activation and deactivation of optical computing devices 22. Optical computing devices 22 each include a transmitter and receiver (transceiver, for example) (not shown) that allows bi-directional communication over communications link 26 in real-time. In certain exemplary embodiments, optical computing devices 22 will transmit all or a portion of the temperature and/or sample characteristic data to CPU station 24 for further analysis. However, in other embodiments, such analysis is completely handled by each optical computing device 22 and the resulting data is then transmitted to CPU station 24 for storage or subsequent analysis. In either embodiment, the processor handling the computations analyzes the temperature/characteristic data and, through utilization of Equation of State (“EOS”) or other optical analysis techniques, derives the temperature and/or characteristic indicated by the transmitted data, as will be readily understood by those ordinarily skilled in the art having the benefit of this disclosure.

Still referring to the exemplary embodiment of FIG. 1, optical computing devices 22 are positioned along workstring 21 at any desired location. In this example, optical computing devices 22 are positioned along the outer diameter of downhole tool 18. Optical computing devices 22 have a temperature and pressure resistant housing sufficient to withstand the harsh downhole environment. A variety of materials may be utilized for the housing, including, for example, stainless steels and their alloys, titanium and other high strength metals, and even carbon fiber composites and sapphire or diamond structures, as understood in the art. In certain embodiments, optical computing devices 22 are dome-shaped modules (akin to a vehicle dome light) which may be permanently or removably attached to a surface using a suitable method (welding, magnets, etc.). Module housing shapes may vary widely, provided they isolate components from the harsh down-hole environment while still allowing a unidirectional or bidirectional optical (or electromagnetic radiation) pathway from sensor to the sample of interest. As will be understood by those ordinarily skilled in the art having the benefit of this disclosure, dimensions would be determined by the specific application and environmental conditions.

Alternatively, optical computing devices 22 may form part of downhole tool 18 (as shown in FIG. 1) along its inner diameter (to detect temperature of fluids flowing through tool 18) or outer diameter (to detect temperature along the annulus between workstring 21 and tubulars 16). In other embodiments, optical computing devices 22 may be coupled to downhole tool 18 using an extendable arm (adjustable stabilizer, casing scraper, downhole tractor, for example) in order to extend optical computing device 22 into close proximity with another surface (casing, formation, etc.) to thereby detect its temperature. As previously described, optical computing devices 22 may also be permanently affixed to the inner diameter of tubular 16 by a welding or other suitable process. However, in yet another embodiment, optical computing devices 22 are removably affixed to the inner diameter of tubulars 16 using magnets or physical structures so that optical computing devices 22 may be periodically removed for service purposes or otherwise.

As mentioned above, those ordinarily skilled in the art having the benefit of this disclosure realize the optical computing devices described herein may be housed or packaged in a variety of ways. In addition to those described herein, exemplary housings also include those described in Patent Cooperation Treaty Application No. ______, filed on Jun. 20, 2013, entitled “IMPLEMENTATION CONCEPTS AND RELATED METHODS FOR OPTICAL COMPUTING DEVICES, the disclosure of which is hereby incorporated by reference in its entirety.

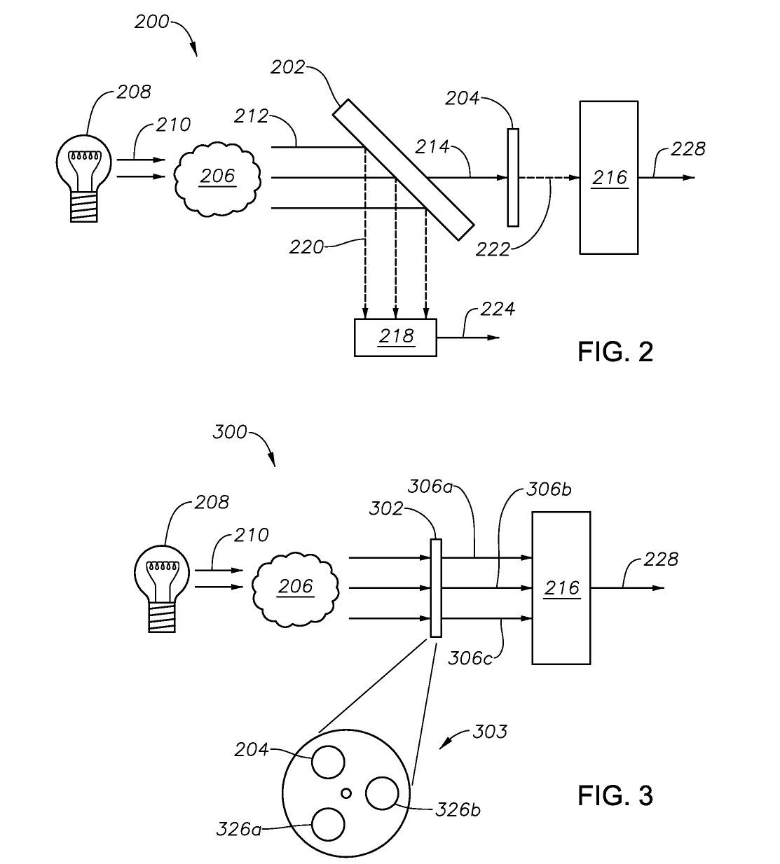

FIG. 2 is a block diagram of an optical computing device 200 employing a transmission mode design, according to certain exemplary embodiments of the present invention. An electromagnetic radiation source 208 may be configured to emit or otherwise generate electromagnetic radiation 210. As understood in the art, electromagnetic radiation source 208 may be any device capable of emitting or generating electromagnetic radiation. For example, electromagnetic radiation source 208 may be a light bulb, light emitting device, laser, blackbody, photonic crystal, or X-Ray source, etc. In one embodiment, electromagnetic radiation 210 may be configured to optically interact with the sample 206 (wellbore fluid flowing through wellbores 12, for example) and generate sample-interacted light 212 directed to a beam splitter 202. Sample 206 may be any fluid (liquid or gas), solid substance or material such as, for example, downhole tool components, tubulars, rock formations, slurries, sands, muds, drill cuttings, concrete, other solid surfaces, etc. In this specific embodiment, however, sample 206 is a multiphase wellbore fluid (comprising oil, gas, water, solids, for example) consisting of a variety of fluid characteristics such as, for example, C1-C4 and higher hydrocarbons, groupings of such elements, and saline water.

Sample 206 may be provided to optical computing device 200 through a flow pipe or sample cell, for example, containing sample 206, whereby it is introduced to electromagnetic radiation 210. Alternatively, optical computing device 200 may utilize an optical configuration consisting of an internal reflectance element which analyzes the wellbore fluid as it flows thereby. While FIG. 2 shows electromagnetic radiation 210 as passing through or incident upon the sample 206 to produce sample-interacted light 212 (i.e., transmission or fluorescent mode), it is also contemplated herein to reflect electromagnetic radiation 210 off of the sample 206 (i.e., reflectance mode), such as in the case of a sample 206 that is translucent, opaque, or solid, and equally generate the sample-interacted light 212.

After being illuminated with electromagnetic radiation 210, sample 206 containing an analyte of interest (a characteristic of the sample, for example) produces an output of electromagnetic radiation (sample-interacted light 212, for example). As previously described, sample-interacted light 212 also contains spectral information that reflects physical variations of the sample due to temperature fluctuations. Ultimately, CPU station 24 (or a processor on-board device 200) analyzes this spectral information in conjunction with baseline spectral information to derive the temperature. Although not specifically shown, one or more spectral elements may be employed in optical computing device 200 in order to restrict the optical wavelengths and/or bandwidths of the system and, thereby, eliminate unwanted electromagnetic radiation existing in wavelength regions that have no importance. As will be understood by those ordinarily skilled in the art having the benefit of this disclosure, such spectral elements can be located anywhere along the optical train, but are typically employed directly after the light source which provides the initial electromagnetic radiation.

Still referring to the exemplary embodiment of FIG. 2, beam splitter 202 is employed to split sample-interacted light 212 into a transmitted electromagnetic radiation 214 and a reflected electromagnetic radiation 220. Transmitted electromagnetic radiation 214 is then directed to one or more optical elements 204. Optical element 204 may be a variety of optical elements such as, for example, one or more narrow band optical filters or ICEs arranged or otherwise used in series in order to determine the characteristics of sample 206. In those embodiments using ICEs, the ICE may be configured to be associated with a particular characteristic of sample 206 or may be designed to approximate or mimic the regression vector of the characteristic in a desired manner, as would be understood by those ordinarily skilled in the art having the benefit of this disclosure. Additionally, in an alternative embodiment, optical element 204 may function as both a beam splitter and computational processor, as will be understood by those same ordinarily skilled persons.

Nevertheless, transmitted electromagnetic radiation 214 then optically interacts with optical element 204 to produce optically interacted light 222. In this embodiment, optically interacted light 222, which is related to the characteristic or analyte of interest, is conveyed to detector 216 for analysis and quantification. In addition to the characteristic or analyte of interest, optically interacted light 22 also contains spectral data utilized to derive temperature. Detector 216 may be any device capable of detecting electromagnetic radiation, and may be generally characterized as an optical transducer. For example, detector 216 may be, but is not limited to, a thermal detector such as a thermopile or photoacoustic detector, a semiconductor detector, a piezo-electric detector, charge coupled device detector, video or array detector, split detector, photon detector (such as a photomultiplier tube), photodiodes, and/or combinations thereof, or the like, or other detectors known to those ordinarily skilled in the art. Detector 216 is further configured to produce an output signal 228 in the form of a voltage that corresponds to the particular temperature and/or characteristic of the sample 206. In at least one embodiment, output signal 228 produced by detector 216 and the temperature/concentration of the characteristic of the sample 206 may be directly proportional. In other embodiments, the relationship may be a polynomial function, an exponential function, and/or a logarithmic function.

Optical computing device 200 includes a second detector 218 arranged to receive and detect reflected electromagnetic radiation and output a normalizing signal 224. As understood in the art, reflected electromagnetic radiation 220 may include a variety of radiating deviations stemming from electromagnetic radiation source 208 such as, for example, intensity fluctuations in the electromagnetic radiation, interferent fluctuations (for example, dust or other interferents passing in front of the electromagnetic radiation source), combinations thereof, or the like. Thus, second detector 218 detects such radiating deviations as well. In an alternative embodiment, second detector 218 may be arranged to receive a portion of the sample-interacted light 212 instead of reflected electromagnetic radiation 220, and thereby compensate for electromagnetic radiating deviations stemming from the electromagnetic radiation source 208. In yet other embodiments, second detector 218 may be arranged to receive a portion of electromagnetic radiation 210 instead of reflected electromagnetic radiation 220, and thereby likewise compensate for electromagnetic radiating deviations stemming from the electromagnetic radiation source 208. Those ordinarily skilled in the art having the benefit of this disclosure will realize there are a variety of design alterations which may be utilized in conjunction with the present invention.

Although not shown in FIG. 2, in certain exemplary embodiments, detector 216 and second detector 218 may be communicably coupled to a signal processor (not shown) on-board optical computing device 200 such that normalizing signal 224 indicative of electromagnetic radiating deviations may be provided or otherwise conveyed thereto. The signal processor may then be configured to computationally combine normalizing signal 224 with output signal 228 to provide a more accurate determination of the temperature and/or characteristic of sample 206. However, in other embodiments that utilized only one detector, the signal processor would be coupled to the one detector. Nevertheless, in the embodiment of FIG. 2, for example, the signal processor computationally combines normalizing signal 224 with output signal 228 via principal component analysis techniques such as, for example, standard partial least squares which are available in most statistical analysis software packages (for example, XL Stat for MICROSOFT® EXCEL®; the UNSCRAMBLER® from CAMO Software and MATLAB® from MATHWORKS®), as will be understood by those ordinarily skilled in the art having the benefit of this disclosure. Thereafter, the resulting data is then transmitted to CPU station 24 via communications link 26 for further operations.

As described herein, the temperature may be determined by a processor on-board optical computing devices 22 or by a processor in CPU station 24. In either embodiment, there are a variety of ways in which to determine the temperature. In one example, output signal 228 comprises spectral data indicative of various physical or chemical characteristics of the sample. Since it is understood that spectral data is contingent upon physical characteristics of the sample, the processor handling the computations will compare the received spectral data with baseline spectral data. Based upon this comparison, the processor maps the computed spectral change to a scale to derive the corresponding temperature. Alternatively, the processor may utilize the spectral shift in the optical element of the computing device itself in order to derive the temperature. In such embodiments, a transducer would not be necessary for calibration of the computing device. Instead, the device may be calibrated before deployment in order to further reduce downhole space requirements.

FIG. 3 illustrates a block diagram of yet another optical computing device 300 employing a time domain mode design, according to certain exemplary embodiments of the present invention. Optical computing device 300 is somewhat similar to optical computing device 200 described with reference to FIG. 2 and, therefore, may be best understood with reference thereto, where like numerals indicate like elements. Optical computing device 300 may include a movable assembly 302 having at least one optical element 204 and two additional optical elements 326a and 326b associated therewith. As illustrated, the movable assembly 302 may be characterized at least in one embodiment as a rotating disc 303, such as, for example, a chopper wheel, wherein optical elements 204, 326a and 326b are radially disposed for rotation therewith. FIG. 3 also illustrates corresponding frontal views of the moveable assembly 302, which is described in more detail below.

Those ordinarily skilled in the art having the benefit of this disclosure will readily recognize, however, that movable assembly 302 may be characterized as any type of movable assembly configured to sequentially align at least one detector with optically interacted light and/or one or more optical elements. Each optical element 204, 326a and 326b may be similar in construction to those as previously described herein, and configured to be either associated or disassociated with a particular temperature and/or characteristic of the sample 206. Although three optical elements are described, more or less optical elements may be employed along movable assembly 302 as desired.

In certain exemplary embodiments, rotating disc 303 may be rotated at a frequency of about 0.1 RPM to about 30,000 RPM. In operation, rotating disc 303 may rotate such that the individual optical elements 204, 326a and 326b may each be exposed to or otherwise optically interact with the sample-interacted light 212 for a distinct brief period of time. Upon optically interacting with the sample-interacted light 212, optical element 204 is configured to generate optically interacted light 306a (a first beam, for example), optical element 326a is configured to generate a second optically interacted light 306b (a second beam, for example) and optical element 326b is configured to generate a normalized electromagnetic radiation 306c (a normalization beam, for example). Detector 216 then receives each beam 306a-c and thereby generates a first, second and third output signal, respectively (output signal 228 comprises the first, second and third signals). Accordingly, a signal processor (not shown) communicatively coupled to detector 216 utilizes the output signal to computationally determine the sample characteristics.

Moreover, in certain exemplary embodiments, detector 216 may be configured to time multiplex beams 306a-c between the individually-detected beams. For example, optical element 204 may be configured to direct first beam 306a toward the detector 216 at a first time T1, optical element 326a may be configured to direct second beam 306b toward the detector 216 at a second time T2, and optical element 326b may be configured to direct third beam 306c toward detector 216 at a third time T3. Consequently, detector 216 receives at least three distinct beams of optically-interacted light which may be computationally combined by a signal processor (not shown) coupled to detector 216 in order to provide an output in the form of a voltage that corresponds to the temperature and/or characteristic of the sample, as previously described. In certain alternate embodiments, beams 306a-c may be averaged over an appropriate time domain (for example, about 1 millisecond to about 1 hour) to more accurately determine the temperature and/or characteristic of sample 206. As previously described, detector 216 is positioned to detect first, second and third beams 306a-c in order to produce output signal 228. In this embodiment, a signal processor (not shown) may be communicably coupled to detector 216 such that output signal 228 may be processed as desired to computationally determine the temperature and/or one or more characteristics of sample 206.

Those ordinarily skilled in the art having the benefit of this disclosure realize the aforementioned optical computing devices are exemplary in nature, and that there are a variety of other optical configurations which may be utilized. These optical configurations not only include the reflection, absorption or transmission methods described herein, but can also involve scattering (Raleigh & Raman, for example) as well as emission (fluorescence, X-ray excitation, etc., for example). In addition, the optical computing devices may comprise a parallel processing configuration whereby the sample-interacted light is split into multiple beams. The multiple beams may then simultaneously go through corresponding ICEs, whereby multiple temperatures and/or analytes of interest are simultaneously detected. The parallel processing configuration is particularly useful in those applications that require extremely low power or no moving parts. In yet another alternate embodiment, various single or multiple ICEs may be positioned in series in a single optical computing device. This embodiment is particularly useful if it is necessary to measure the temperature or concentrations of the analytes in different locations (in each individual mixing pipe, for example). It is also sometimes helpful if each of the ICEs use two substantially different light sources (UV and IR, for example) to cover the optical activity of all the temperatures or analytes of interest (i.e., some analytes might be only UV active, while others are IR active). Nevertheless, those ordinarily skilled in the art having the benefit of this disclosure will realize the choice of a specific optical configuration is mainly dependent upon the specific application and analytes of interest.

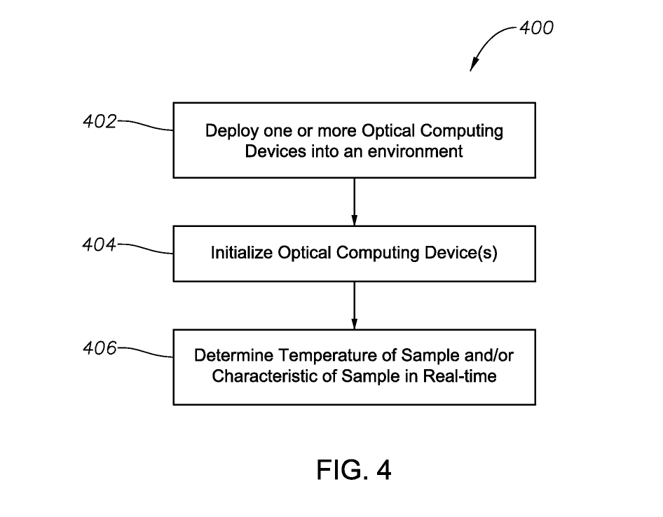

In view of the foregoing description, an exemplary methodology of the present invention will now be described with reference to the flow chart 400 of FIG. 4. As stated throughout this description, the optical computing devices described herein may be utilized to detect temperature in a variety of environments. In one such application at block 402, one or more optical computing devices are deployed in an environment (downhole well, for example) as part of a monitoring system. When it is desired to perform temperature detection, CPU station 24 initializes one or more optical computing devices at block 404. As wellbore fluid or other samples of interest flow through the well and past the activated optical computing devices, the optical elements contained therein optically interact with the radiation emanating from the sample to acquire and determine the temperature of the sample at block 406. Alternatively, at block 406, the optical computing device (or the CPU station) may also utilize the radiation emanating from the sample to determine one or more characteristics of the sample (presence of C1-C4 hydrocarbon, for example). The determination of block 406 may be performed in real-time by the optical computing device itself or temperature/characteristic data is generated by the computing device and transmitted to the CPU station for further processing in real-time.

In certain other exemplary embodiments, temperature data from the optical computing device can be utilized locally in the well at the device or transmitted to the surface or other remote data processing equipment inside or outside the well to trigger alert signals based on predetermined criteria, such as, for example, temperature limits. Crossing these boundary limits may trigger alerts and remedial actions to correct further temperature increases, process deficiencies, or conditions. Examples include, but are not limited to, the following: operator alerts at surface, automated valve actuation at surface or down hole to alter flow conditions, trigger/control of additional injection fluids and chemicals for treatments and control of scale and other unwanted conditions.

Accordingly, the present invention provides an optical computing device that determines and monitors temperature in real-time by deriving the data directly from the output of an optical element. The monitored temperatures may correspond to wellbore fluids, various downhole tools (e.g., electrical submersible pumps), well zones, etc. The ability to measure physical and environmental changes in real-time, independent of the optical computing device's primary function provides great advantage because, in addition to characteristic data, temperature data can be collected and transmitted with the original signal without the need for additional equipment, such as gauges or transducers. In addition, more elaborate mapping of the formation can be achieved through use of a plurality of downhole optical computing devices. Moreover, if data verification or calibration is a requirement of the application, the temperature data can serve as a comparator to the characteristic data or vice versa. Accordingly, the present invention provides the ability to monitor temperature in real-time using a low-cost and highly compact methodology.

An exemplary embodiment of the present invention provides a method utilizing an optical computing device to determine temperature of a sample, the method comprising deploying an optical computing device into an environment, the optical computing device comprising an optical element and a detector; optically interacting electromagnetic radiation with a sample to produce sample-interacted light; optically interacting the optical element with the sample-interacted light to generate optically-interacted light which corresponds to a characteristic of the sample; generating a signal that corresponds to the optically-interacted light through utilization of the detector; and determining a temperature of the sample using the signal. In another, the environment is a wellbore. In yet another, the optical element is an Integrated Computational Element.

In another, the temperature of the sample is determined in real-time. In yet another, the method further comprises generating the electromagnetic radiation using an electromagnetic radiation source. In another, the electromagnetic radiation emanates from the sample. In another, determining the temperature of the sample is achieved using a signal processor communicably coupled to the detector. In yet another, deploying the optical computing device further comprises deploying the optical computing device as part of a downhole tool or casing extending along a wellbore. In another, the method further comprises generating an alert signal in response to the temperature of the sample.

An exemplary embodiment of the present invention provides an optical computing device to determine temperature of a sample, comprising electromagnetic radiation that optically interacts with a sample to produce sample-interacted light; a first optical element that optically interacts with the sample-interacted light to produce optically-interacted light which corresponds to a characteristic of the sample; and a detector positioned to measure the optically-interacted light and thereby generate a signal utilized to determine a temperature of the sample. In another, the sample is at least one of a wellbore fluid, downhole tool or rock formation. In yet another, the computing device further comprises an electromagnetic radiation source that generates the electromagnetic radiation. In another, the electromagnetic radiation is radiation emanating from the sample.

In yet another, the computing device further comprises a signal processor communicably coupled to the detector to computationally determine the temperature of the sample in real-time. In another, the optical element is an Integrated Computational Element. In yet another, the characteristic of the sample is at least one of a C1 hydrocarbon, C2 hydrocarbon, C3 hydrocarbon or C4 hydrocarbon. In another, the optical computing device comprises part of a downhole tool or casing extending along a wellbore.

Another exemplary methodology of the present invention provides a method utilizing an optical computing device to determine temperature of a sample, the method comprising deploying an optical computing device into an environment; and determining a temperature of the sample present within the environment using the optical computing device. In another, the environment is a wellbore. In yet another, the optical element is an Integrated Computational Element.

Although various embodiments and methodologies have been shown and described, the invention is not limited to such embodiments and methodologies, and will be understood to include all modifications and variations as would be apparent to one ordinarily skilled in the art. Therefore, it should be understood that the invention is not intended to be limited to the particular forms disclosed. Rather, the intention is to cover all modifications, equivalents and alternatives falling within the spirit and scope of the invention as defined by the appended claims.

What is claimed is:

1. A method utilizing an optical computing device to determine temperature of a sample, the method comprising:
deploying an optical computing device into an environment, the optical computing device comprising an optical element and a detector;
optically interacting electromagnetic radiation with a sample to produce sample-interacted light;
optically interacting the optical element with the sample-interacted light to generate optically-interacted light which corresponds to a characteristic of the sample;
generating a signal that corresponds to the optically-interacted light through utilization of the detector; and
determining a temperature of the sample using the signal.

2. A method as defined in claim 1, wherein the environment is a wellbore.

3. A method as defined in claim 1, wherein the optical element is an Integrated Computational Element.

4. A method as defined in claim 1, wherein the temperature of the sample is determined in real-time.

5. A method as defined in claim 1, further comprising generating the electromagnetic radiation using an electromagnetic radiation source.

6. A method as defined in claim 1, wherein the electromagnetic radiation emanates from the sample.

7. A method as defined in claim 1, wherein determining the temperature of the sample is achieved using a signal processor communicably coupled to the detector.

8. A method as defined in claim 1, wherein deploying the optical computing device further comprises deploying the optical computing device as part of a downhole tool or casing extending along a wellbore.

9. A method as defined in claim 1, further comprising generating an alert signal in response to the temperature of the sample.

10. An optical computing device to determine temperature of a sample, comprising:
electromagnetic radiation that optically interacts with a sample to produce sample-interacted light;
a first optical element that optically interacts with the sample-interacted light to produce optically-interacted light which corresponds to a characteristic of the sample; and
a detector positioned to measure the optically-interacted light and thereby generate a signal utilized to determine a temperature of the sample.

11. An optical computing device as defined in claim 10, wherein the sample is at least one of a wellbore fluid, downhole tool or rock formation.

12. An optical computing device as defined in claim 10, further comprising an electromagnetic radiation source that generates the electromagnetic radiation.

13. An optical computing device as defined in claim 10, wherein the electromagnetic radiation is radiation emanating from the sample.

14. An optical computing device as defined in claim 10, further comprising a signal processor communicably coupled to the detector to computationally determine the temperature of the sample in real-time.

15. An optical computing device as defined in claim 10, wherein the optical element is an Integrated Computational Element.

16. An optical computing device as defined in claim 10, wherein the characteristic of the sample is at least one of a C1 hydrocarbon, C2 hydrocarbon, C3 hydrocarbon or C4 hydrocarbon.

17. An optical computing device as defined in claim 10, wherein the optical computing device comprises part of a downhole tool or casing extending along a wellbore.

18. A method utilizing an optical computing device to determine temperature of a sample, the method comprising:
deploying an optical computing device into an environment; and
determining a temperature of the sample present within the environment using the optical computing device.

19. A method as defined in claim 18, wherein the environment is a wellbore.

20. A method as defined in claim 18, wherein the optical element is an Integrated Computational Element.
